(19) United States
(12) Patent Application Publication
(10) Pub. No.: US 2024/0315861 A1
(43) Pub. Date: Sep. 26, 2024

(54) HIGH ANKLE BRACE

(71) Applicants: Martha Hall, Landenberg, PA (US); Brandon Desantis, Newark, DE (US); Kiki Bink, Wageningen (NL); Riley Curtin, Hershey, PA (US); Andrew Reynolds, Newark, DE (US); Rachel Swamy, Newark, DE (US); Jaeah Yoo, Newark, DE (US); Marjan Ashrafi, Newark, DE (US)

(72) Inventors: Martha Hall, Landenberg, PA (US); Brandon Desantis, Newark, DE (US); Kiki Bink, Wageningen (NL); Riley Curtin, Hershey, PA (US); Andrew Reynolds, Newark, DE (US); Rachel Swamy, Newark, DE (US); Jaeah Yoo, Newark, DE (US); Marjan Ashrafi, Newark, DE (US)

(73) Assignee: University of Delaware, Newark, DE (US)

(21) Appl. No.: 18/275,069

(22) PCT Filed: Feb. 2, 2022
(86) PCT No.: PCT/US2022/014909

§ 371 Date: Jul. 31, 2023

Related U.S. Application Data
(60) Provisional application No. 63/144,531, filed on Feb. 2, 2021.

(51) Int. Cl. A61F 5/01

(52) U.S. Cl. CPC A61F 5/0111

(57) ABSTRACT

An ankle brace configured to be worn by a target wearer having a foot, an ankle, a lower leg, and a tibia and a fibula disposed within the lower leg, is disclosed. The ankle brace comprises a foot plate having a foot bed contoured to underlie the foot. The foot bed has a heel cup extending upward from the foot bed and has a relatively greater flexibility than a front foot portion of the foot bed. An orthopedic splint is pivotably coupled to the foot plate and configured to at least partially wrap around the lower leg. The orthopedic splint is disposed in an adjustable pocket, which is configured to adjust compression of the orthopedic splint relative to the lower leg to promote a rehabilitative position of the tibia and the fibula. The foot plate may be fabricated with a computer aided manufacturing process.

### CROSS REFERENCE TO RELATED APPLICATIONS

This application claims priority from U.S. Provisional Application Ser. No. 63/144,531, titled CUSTOMIZED HIGH ANKLE BRACE, filed Feb. 2, 2021, incorporated herein by reference in its entirety.

### STATEMENT REGARDING FEDERALLY SPONSORED RESEARCH

This invention was made with government support under R25 EB014790 awarded by the National Institutes of Health. The government has certain rights in the invention.

### FIELD OF THE INVENTION

The present invention relates generally to orthopedic and orthotic devices, and more particularly, to ankle and foot braces for use in proper care and is management of injuries and impairments related to the ankle.

### BACKGROUND OF THE INVENTION

High ankle sprains (syndesmotic sprains) occur when there is stretching of and damage to the proximal ankle ligaments, which hold the bones of the lower leg (e.g. tibia and fibula) in place and prevent separation during weight bearing activities. In particular, the stretching of the stabilizing ankle ligaments by forceful rotation of the ankle and foot beyond their normal range of motion is the most common cause of injury for high ankle sprain. These high ankle sprains are prevalent in contact sports, especially football, due to the high probability of rotational injuries. Studies suggest that high ankle sprains account for 50-75% of all ankle sprains diagnosed in contact sports.

It will be appreciated that throughout this specification the term “high ankle sprain” includes pain experienced or focused above the ankle, accompanied by tenderness in the distal lower leg. As a result of a high ankle sprain, at least the anterior inferior tibiofibular ligament is weakened, causing the talus bone to wedge apart the tibia and fibula. The separation of these bones can cause severe pain in the weight-bearing region. Due to the weakness in the proximal ankle ligaments, there is an increased likelihood of re-injury to the medial and lateral ankle ligaments. Further, the recovery time for high ankle sprains can be considerably longer than other ankle sprains, e.g., with an average of six weeks before an athlete can to return to play. This long recovery time and ankle-foot joint instability becomes problematic for the patient (e.g. athlete) and the training staff.

Further, the term “rehabilitative position” refers to the rehabilitation and injury management of high ankle sprains. The rehabilitative position includes appropriate treatment measures provided to the ankle and can be utilized throughout the duration of the recovery process. Specifically, the “rehabilitative position of the tibia and fibula” includes properly aligning and/or maintaining the tibia and fibula in the correct position to promote the healing and regeneration of the ankle ligaments, chiefly through minimizing the rotation of the ankle. Minimizing the rotation of the ankle is achieved by effectively preventing the separation of these bones (e.g. tibia and fibula) and reducing risk of re-injury, while allowing adequate mobility for competitive play.

With reference to the drawings, FIGS. 1A-1E are images of examples of prior art ankle brace devices and techniques, including a walking boot (a), a stirrup-like brace (b), and (c) a common ankle brace for lateral and medial ankle sprains. The is assistive walking booth (a) can be used in the early stages of high ankle sprain recovery process to reduce weight-bearing and to increase tibia-fibula compression and stability. However, this boot cannot be used during athletic activities, including competitive play, and so offers limited use as a preliminary protective device only. The stirrup-like brace (b), so called because the brace resembles a riding stirrup, protects the proximal ankle ligaments using inflatable air pumps and circumferential support. This brace is typically employed when the patient begins their return to the athletic activity or sport and is used for light, everyday activity. The lateral and medial ankle sprain braces (c), such as the ASO ankle brace designed and manufactured by Medical Specialties Incorporated of Charlotte, North Carolina, typically combine lacing, figure-8 straps, and circumferential Velcro® bands. Other prior art ankle braces include those designed and manufactured by Ultra Ankle of Indianapolis, Indiana; DonJoy Performance of Dallas, Texas; Ossur of Orange County, California; and Acumed of Hillsboro, Oregon. However, prior art braces do not sufficiently compress the ligaments above the ankle, e.g. the anterior inferior tibiofibular ligament (AITFL) and the posterior inferior tibiofibular ligament (PITFL), during the recovery process for high ankle sprain, and therefore fall short in stabilizing the ankle joint.

Further, ankle braces are generally viewed as inefficient in some athletic activities, such as high contact sports, because they are not sufficiently durable to withstand frequent use and/or the rigor of the activity for the full duration of the season. Thus, other prior art stabilizing techniques may be used, including taping methods (d) having the standard figure-8 taping of the lateral and medial ligaments to limit inversion or eversion of the ankle, and also having the circumferential taping around the distal tibia and fibula bones about 3 inches superior to the ankle, as shown in (d) of FIG. 1. However, taping is only implemented after completion of the rehabilitation process, and so fails to accelerate return of athlete or patient to (competitive) play. Second, taping is ineffective at reducing the pain of weight bearing activities, as is it difficult to sufficiently secure the tibiofibular junction so as to not stretch the injured ligaments. Lastly, taping takes time for both the patient (e.g. athlete) and the sports staff (e.g. trainer) or medical professional and must be redone before each session of athletic activity. This re-taping can create inconsistency relative to use of a standardized brace.

Finally, prior art ankle braces may also include a custom fit orthotic (e), such as the specialized brace designed and manufactured by Independence Prosthetics and Orthotics Inc. (IPO) of Newark, Delaware. The brace (e) of FIG. 1 can be molded using standard casting techniques. The brace (e) may include a layer of leather over the plastic for comfort and durability, as well as leather laces and a circumferential is Velcro® strap to allow for tightening and an adjustable fit. However, the brace (e) remains bulky, difficult to use, and cost prohibitive for repeated and/or widespread use.

Thus, improved systems and devices are desired for improving rehabilitation and health outcomes by maintaining the proper position and movement of the tibia and the fibula during post ankle injury care and management. Improved systems and devices are desired for improving rehabilitation and health outcomes of ankle injury patients by minimizing internal and external rotation of the proximal ankle joint and compressing the target ligaments (e.g., the anterior inferior tibiofibular ligament (AITFL) and the posterior inferior tibiofibular ligament (PITFL)) during the recovery process, such that patients can experience reduced pain, reduced joint instability, and shorter recovery times.

### SUMMARY OF THE INVENTION

Aspects of the present invention are directed to simulation systems and devices for use in neonatal care.

In accordance with one aspect of the present invention, an ankle brace configured to be worn by a target wearer, the target wearer having a foot, an ankle, a lower leg, and a tibia and a fibula disposed within the lower leg, is disclosed. The ankle brace comprises a foot plate having a foot bed contoured to underlie the foot of the target wearer. The foot bed has a heel cup extending generally upward from the foot bed and along a posterior aspect of the ankle of the target wearer. The heel cup has a relatively greater flexibility than a front foot portion of the foot bed. The ankle brace further comprises an orthopedic splint pivotably coupled to the foot plate and configured to at least partially wrap around the lower leg of the target wearer. The orthopedic splint is disposed in an adjustable pocket and the adjustable pocket is configured to adjust compression of the orthopedic splint relative to the lower leg of the wearer to promote a rehabilitative position of the tibia and the fibula of the target wearer.

### BRIEF DESCRIPTION OF THE DRAWINGS

The invention is best understood from the following detailed description when read in connection with the accompanying drawings, with like elements having the same reference numerals. When a plurality of similar elements are present, a single reference numeral may be assigned to the plurality of similar elements with a small letter designation referring to specific elements. When referring to the elements collectively or to a non-specific one or more of the elements, the small letter designation may be dropped. This emphasizes that according to common practice, the various features of the drawings are not drawn to scale unless otherwise indicated. On the contrary, the dimensions of the various features may be expanded or reduced for is clarity. Included in the drawings are the following figures:

FIGS. 1A-1E are images depicting ankle braces in accordance with prior art;

FIG. 2 is an image depicting an exemplary ankle brace;

FIGS. 3A-3B are images depicting an exemplary foot plate;

FIG. 4 shows an exemplary orthopedic splint;

FIGS. 5A-5B are images depicting the orthopedic splint comprising moldable thermoplastic material;

FIGS. 6A-6B are images an exemplary compression sleeve;

FIGS. 7A-7D are images depicting the compression sleeve having at least one strap, a corresponding buckle receiving the strap, and a ratchet;

FIG. 8 is an image depicting another exemplary ankle brace;

FIG. 9 is an image depicting another exemplary foot plate and another exemplary orthopedic splint; and

FIG. 10 is an image depicting integration of an exemplary ankle brace with a shoe.

FIGS. 11A-11C depict exemplary details of an exemplary pocket for receiving the orthopedic splint.

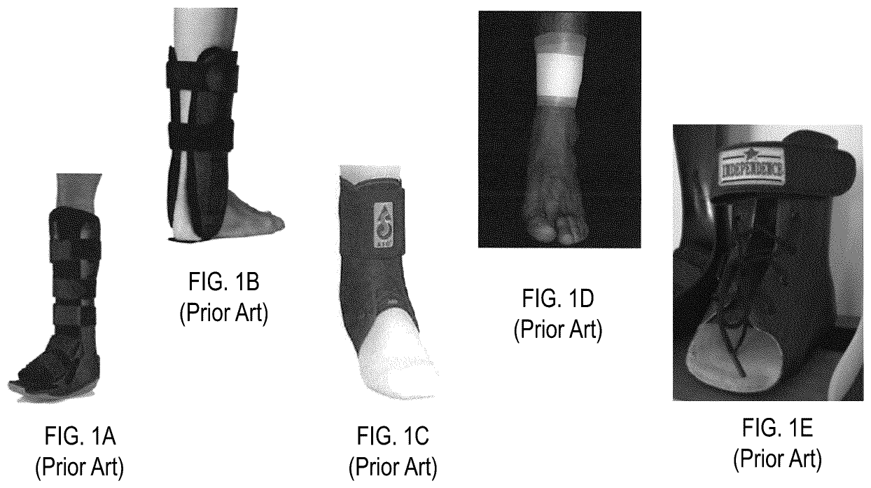

### DETAILED DESCRIPTION OF THE INVENTION

Aspects of the invention are described herein with reference to care and management of ankle injuries, such as high ankle sprain. It will be understood by one of ordinary skill in the art that the exemplary braces described herein are not limited to specific patients who have ankle injuries or to specific care and management protocols. Other types of patients or medical treatment plans suitable for use with the disclosed braces will be known to one of ordinary skill in the art from the description herein.

A “target wearer” as described herein is a wearer having characteristics at least within a known or expected range of values, which may be actual values as measured, or expected ranges based upon birth gender, height, weight, and other physiological factors. It should be understood that designs intended for a target wearer may be sized to accommodate a specific individual wearer or a spectrum of wearers having physiological measurements within the expected ranges of values.

While the exemplary embodiments of the invention are described herein with respect to athletic activities, such as contact sports (e.g. football), it will be understood that the invention is not so limited. Suitable applications for systems of the present invention include, for example, ankle and foot braces for use in military gear, police gear, and construction gear. Other suitable applications will be readily understood by one of ordinary skill in the art from the description herein.

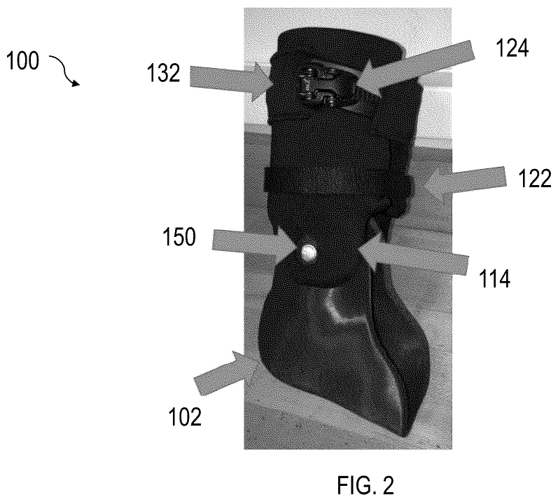

FIG. 2 illustrates an example ankle brace 100 in accordance with aspects of the present invention. Ankle brace 100 is usable for proper care of injuries and impairments related to the ankle. The ankle brace 100 can be adjusted to provide proper care of various target wearers (e.g. patients, athletes, etc.) and to adapt to various scenarios (e.g. during competitive play, performing day-to-day activities, etc.).

In general, ankle brace 100 is configured to be worn by a target wearer, who has a foot, an ankle, a lower leg, and a tibia and a fibula disposed within the lower leg. Ankle brace 100 generally includes a foot plate 102 and an orthopedic splint 114. The high ankle brace 100 is adapted to provide a custom fit to the target wearer, while avoiding the excessive cost and casting process associated with custom orthotics or orthopedic devices. Additional details of ankle brace 100 are described below.

The ankle brace 100 comprises a foot plate 102, which may be configured to be lightweight, but durable. In an exemplary embodiment, the foot plate 102 may have a foot bed 104 contoured to underlie at least a portion of the foot of the wearer. Specifically, the foot bed 104 may have a front edge that does not extend beyond the metatarsal bone heads of the target wearer's foot. Additionally or optionally, the front edge of the foot bed 104 may be disposed within an area defined between the metatarsal bone heads and the tarsometatarsal joints of the target wearer's foot.

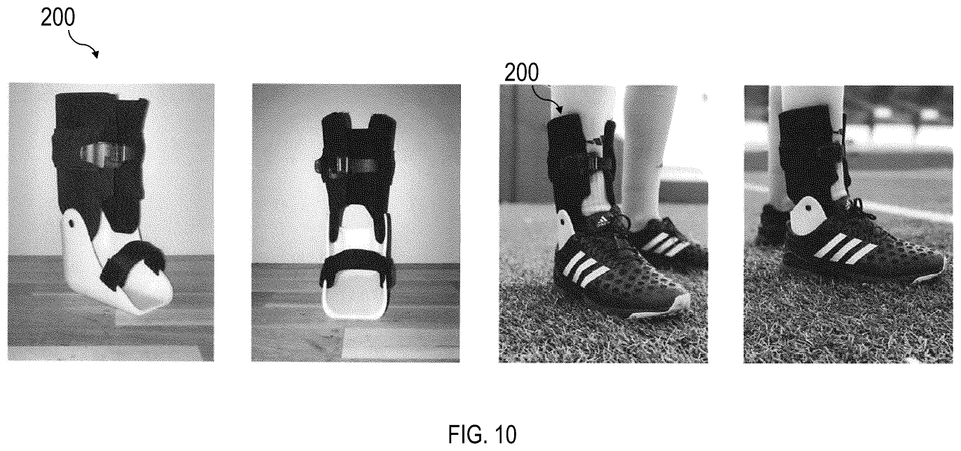

Further, the foot plate 102 is ergonomically designed to be easily integrated within the target wearer's shoe, particularly on the insole of the target wearer's shoe (as best shown, for example, in FIG. 10). This ergonomic design can be achieved by having a contoured foot bed 104, for example. The contoured surface of the foot bed 104 may be customizable based on the anatomy and/or physiological characteristics of the target wearer (e.g. patient, athlete, etc.). Providing a custom-fitted foot plate 102 is configured to minimize the internal rotation of the ankle (pronation) and/or external rotation of the ankle (supination). In addition, providing a custom fit can desirably reduce the size and increase the comfortability of the high ankle brace 100.

In one non-limiting example, the use of 3D printing techniques can permit at least the foot plate 102 to be custom fitted to the target wearer's foot and offer a completely customizable ankle brace 100, without relying on standard and expensive casting techniques. Forming the foot plate 102 with the use of 3D printing techniques can offer a custom fit, where multiple iterations and sizing alterations can be made relatively quickly and at a low cost. As used herein and throughout the specification, the term “custom fit” is not limited to a user-by-user (e.g. target wearer) basis, but can also refer to providing a relatively smaller number of standard is incremental sizes (S, M, L, XL, 2XL) of the ankle brace 100, which correspond to US shoe sizes. The ankle brace 100 of a standard incremental size (e.g. XS) may provide additional customization (or further custom fit) by including at least one adjustable component. One skilled in the art would understand from the description herein that the adjustable component may be configured for permitting adjustments that result in more individualized or custom fit.

In particular, the contours of the foot plate 102 having a foot bed 104, may be have a custom fit by digitally capturing an image of the foot (e.g., using a 3D scanned image of the foot) and then providing a size and contour of the foot plate 102 based on this digital model. Additionally or optionally, the overall shape of the brace 100 may be customized by digitally capturing an image of the foot and ankle of the target wearer (e.g., using a 3D scanned image of the foot and the ankle) and then providing the size and contour of the brace 100 based on this digital model. One skilled in the art would understand from the description herein that the digital capture process may include use of a 3D scanning or imaging program, such as the Skanect 3D Scanning Software, as described and designed by Occipital Inc of Boulder, Colorado.

After digitally capturing the image of the foot and/or ankle of the wearer, the digital image or model is subsequently converted into a file usable in a multi-purpose 3D sculpting design platform, such as Geomagic® Freeform®, and a haptic styling device can be used to sculpt the foot plate 102 around the captured image of the foot and/or ankle of the wearer. In a non-limiting example, sculpting the foot plate 102 based on the digital image of the foot may be guided by the following parameters. Although the parameters provide a degree of standardization in the sculpting process, one skilled in the art would nevertheless understand from the description herein that the sculpting process will vary based on the anatomy of the wearer, the skill of the sculptor, variations in the sculpting program used, or combinations thereof.

From a plantar view of the digital image of the foot, a curve must be drawn on the left side of the foot. Said curve must be drawn slightly proximal with respect to the metatarsal bone heads, and must not go beyond or pass the tarsometatarsal joint. From the medial view of the digital image of the foot, the drawn curve continues to extend around the medial malleolus of the ankle. From the anterior view of the digital image of the foot, the drawn curve continues to extend around the ankle joint, such that at least the calcaneus portion of the heel is covered. In an exemplary embodiment, the foot plate 102 includes an enclosed calcaneal heel cup 106. From the lateral view of the digital image of the foot, the drawn curve continues to extend around the lateral malleolus of the ankle, such that the curve around the medial malleolus and lateral malleolus are generally symmetrical.

Subsequently, after designing/sculpting a digital model of the foot plate 102 based on the digitally captured image of the foot and/or ankle, a 3D printing technique is used to construct the customized foot plate 102. The 3D printing technique may comprise steps of (i) using a 3D printer, such as Lulzbot Taz 6 Dual Extruder v3, (ii) using a 3D printing element, such as for example a thermoplastic polymer, such as the Cheetah® 3D PRINTER FILAMENT (2.85 mm), as designed and described by NinjaTek of Manheim, Pennsylvania, and (iii) using a support material, such as polyvinyl alcohol (PVA). Other 3D printers, 3D printing elements, and support materials will be known to one of ordinary skill in the art from the description herein. Specific settings may include the infill at 50% and support placement at “Touching Buildplate.”

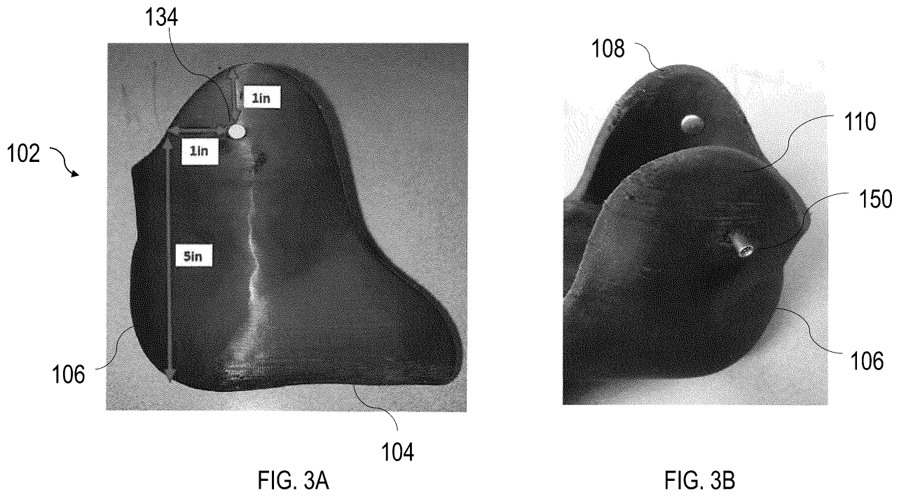

Referring now to FIGS. 3A-3B, the foot plate 102 includes a medial wing 108 extending generally upward from the foot bed 104 and along a medial aspect of the wearer's ankle. In an exemplary embodiment, the medial wing 108 extends around the medial malleolus of the ankle. The foot plate also including a substantially identical lateral wing 110 extending generally upward from the foot bed 104 and along a lateral aspect of the wearer's ankle. In an exemplary embodiment, the lateral wing 110 extends around the lateral malleolus of the ankle. The medial wing 108 and the lateral wing 110 comprise Cheetah® 3D PRINTER FILAMENT (2.85 mm), as designed and described by NinjaTek of Manheim, Pennsylvania, in order to achieve the strong structural integrity required to provide medial and lateral support to the ankle joint.

The foot plate 102 includes the heel cup 106 for further stabilizing the ankle joint. The heel cup 106 stabilizes the ankle by preventing forceful external rotation of the ankle beyond a normal range of motion. To achieve this, the heel cup 106 extends generally upward from the foot bed 104 and along a posterior aspect of the ankle of the wearer. In an exemplary embodiment, the heel cup 106 extends upward from the food bed 104 such that at least the calcaneus portion of the wearer's heel is partially or completely covered (i.e. enclosed). In an exemplary embodiment, the heel cup 106 extends upwardly for a distance of no more than 6 inches from the foot bed 104, but one skilled in the art would understand that the heel cup may extend upwardly for a distance that varies based on one or more physiological characteristics of the target wearer, such as height and shoe size.

Still further, as best shown in FIGS. 2, 6B, and 7B, this configuration of the foot plate 102 as including the lateral wing 110, the medial wing 108, and the heel cup 106 forms a generally open (i.e., unimpeded by other materials independent of the brace 100 or other components of the brace 100) front foot portion and front ankle portion to facilitate overall mobility of the wearer (e.g. walking, running, etc.), especially during play or a sport scenario. As will be discussed later, the area between is the front ankle and front foot portions are left substantially open to facilitate the articulation or pivoting movement of the foot plate 102 relative to the orthopedic splint 114.

The material properties (e.g. shape, thickness of materials, etc.) of the foot plate 102 was optimized, in part, via a Finite Element Analysis (FEA) used to assess the behavior of components of the brace 100 when a load is applied. The applied load may comprise pressure, force, temperature, gravity, centrifugal loads, or combinations thereof. In particular, the applied loads simulated the internal and external forces that the brace 100 may experience during impact situations that can increase risk of high ankle sprains, including but not limited to participation in contact sports or other athletic activities. The results of the FEA indicated that the heel cup 106 enclosing the calcaneal portion of the wearer's foot more effectively distributes the simulated loads compared to heel cups having a relatively different (e.g. lower) profile, i.e., shorter distance between an edge of the heel cup 106 and the foot bed 104. Thus, having an enclosed calcaneal heel cup or plate 106 allows for optimal stabilization of the ankle joint, while simultaneously reducing risk of failure of the brace 100.

The heel cup 106 has a relatively greater flexibility than a front foot portion of the foot bed 104. In an exemplary embodiment, the heel cup 106 comprises a first set of material properties and the front foot portion has a second set of material properties that are different than the first set of material properties. Material properties include, but are not limited to, properties related to flexibility. This non-uniformity in flexibility between the front foot portion and the heel cup 106 can facilitate mobility of the wearer (e.g. walking, running, etc.), while simultaneously stabilizing the ankle joint. In particular, the geometry of the heel cup 102 is critical in providing sufficient compressions of the target ligaments above the ankle in the syndesmosis (as will be discussed below). These target ligaments, including the anterior inferior tibiofibular ligament (AITFL) and the posterior inferior tibiofibular ligament (PITFL), are overlooked in existing lateral and medial ankle sprain braces, which thus fall short in stabilizing the ankle during proper care of high ankle sprains (including preventing or reducing likelihood of re-injury that can prolong recovery time).

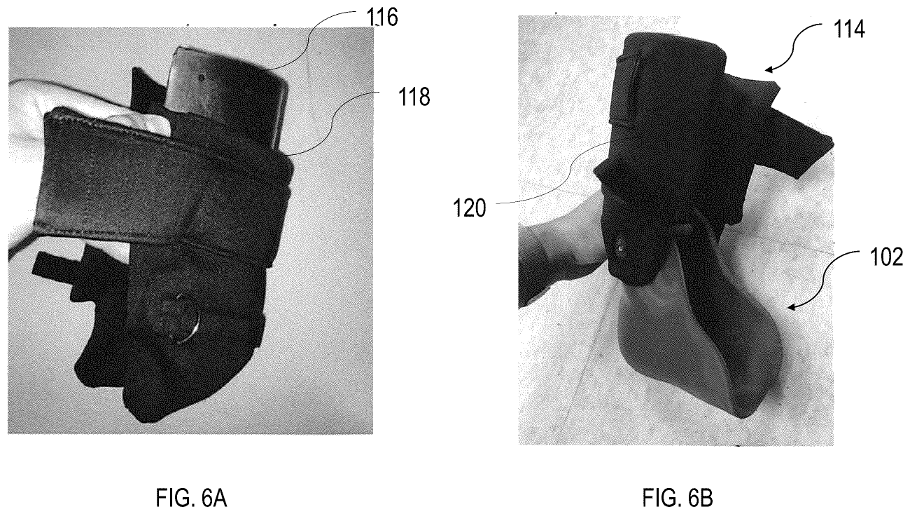

As shown in FIGS. 2 and 6B, the orthopedic splint 114 is pivotably coupled to the foot plate 102. In an exemplary embodiment, the orthopedic splint 114 is pivotably coupled to the foot plate 102 via a hinge mechanism adapted to withstand the impacts and forces experienced by the brace 100 during each use cycle and/or over time (e.g. for a full sports season of approximately 5 months).

In one example, a hinge design found in existing hinged ankle-foot orthosis (AFO) braces comprises an intermediary piece for connecting a lower leg is section and a foot plate of the AFO brace. The intermediary piece comprises flexible material, thereby allowing the lower leg section to articulate relative to the foot plate of the AFO brace. However, this AFO brace hinge increases the likelihood of creating an undesirable protrusion (where there are overlapping structures, e.g. portion of the foot plate, portion of the lower leg section, and the intermediary connecting piece) extending outwardly from the brace. This configuration may also decrease ease of integration with the target wearer's shoe (as it creates bulk and an uncomfortable contact point), and further may define a weak structural point against protecting the ankle from impact or force encountered during play or a sports scenario.

Unlike the problems posed by the AFO brace hinge, embodiments of the present invention include an orthopedic splint 114 pivotably coupled to the foot plate 102 via use of double-capped metal (e.g. steel) rivets 150, which desirably permits ease of assembly and disassembly of the components of ankle brace 100. Further, the application of a sealer, including but not limited to Loctite® Threadlocker Red 271™, may be used to strengthen the fastening functionality of the rivets 150. This application of a sealer may help ensure that the rivets 150 do not inadvertently separate from each other, especially during and after repeated articulation of the foot plate 102 relative to the orthopedic splint 114 during rigorous activities, such as sports. The rivets 150 may still be removeable (i.e. to facilitate easy cleaning, or replacement of parts, etc.), but successful separation of the foot plate 102 from the orthopedic splint 114 may be made possible only upon application of a considerable amount of force. Although there remains a small risk that the rivets 150 may loosen over time, especially after repeated use, the application of a sealer, such as Loctite Loctite® Threadlocker Red 271™, is relatively easy to re-apply, as appropriate. For additional security, in one embodiment, washers were incorporated to ensure the attachment provided by rivets 150.

During assembly of the foot plate 102 and the orthopedic splint 114, as shown in FIG. 3A, the attachment points 134 (e.g. holes) of the foot plate 102 for the rivets 150 and the attachment points 136 (e.g. holes) of the splint 114 for the rivets 150 may be located at the lateral wing 110 and medial wing 108 of the foot plate 102, and a lower portion of the orthopedic splint 114, respectively. However, it should be understood that the illustrated locations (as depicted by measurements of distances in inches) of the attachment points 134, 136 are not intended to be limiting, such that they may be positioned to accommodate one or more physiological characteristics of the target wearer (e.g. height, shoe size, etc.). In an exemplary embodiment, the foot plate 102 is positioned between the target wearer's lower leg and the orthopedic splint 114, such that each of the lateral wing 110 and medial wing 108 extends along an is interior surface of the orthopedic splint 114. The foregoing construction of foot plate 102, which may be a custom fit component, allows for a more snug fit around the ankle joint, thereby more efficiently compressing the target ligaments and provides more effective stabilization of the ankle.

The durability (i.e. ability to withstand both long term use and direct impact forces, as well as the repeated range of motion) of the hinge mechanism of the prototype brace 100 was assessed using testing protocols detailed in the example further below. In summary, the assessment involved repeated movement of the brace 100 from a desired plantar flexed angle to a desired dorsiflex angle, at a speed of about 50 times per minute for 90 minutes to achieve over 4,000 repetitions. The assessment indicated that the brace only slightly loosened, but did not break, falter, deform, or otherwise became inoperable or unsuitable for use during competitive play or other athletic activities. Accordingly, the hinge mechanism was assessed to receive a preliminary passing score.

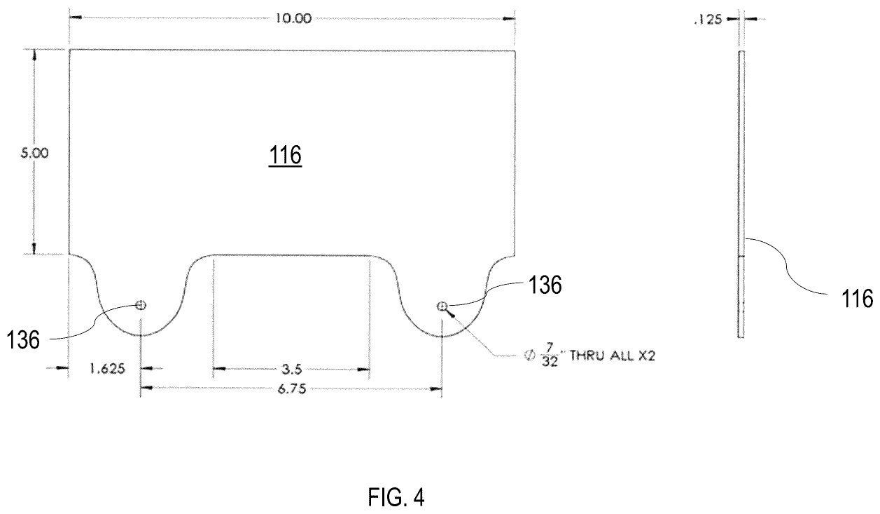

Referring now to FIGS. 4, 5A-5B, and 6A-6B, the orthopedic splint 114 is further configured to at least partially wrap around the lower leg of the target wearer. The orthopedic splint 114 may comprise moldable thermoplastic material 116, such as a pre-cut and planar sheet fabricated from ORTHOPLAST® thermoplastic, as designed and manufactured by Johnson & Johnson of New Brunswick, New Jersey. Preferably, the thermoplastic material 116, e.g. ORTHOPLAST® thermoplastic, is relatively rigid (as compared to other components of the brace, such as an adjustable pocket 118 (discussed below)), and has the desired strength to promote a rehabilitative position of the tibia and fibula of the target wearer, i.e. prevent separation of the fibula and tibia. Further, the ability of the brace 100 to protect the ankle joint against the force of impacts (e.g. during play) is desired. Nonetheless, it is also desirable that material 116 be sufficiently thin to enable comfort, flexibility, and ease of movement during athletic activities or day-to-day life. To this end, the material 116 may be limited in thickness (as shown in FIG. 4) to a range that provides protection against high impact forces without being overly thick.

The thermoplastic material 116 may be pre-cut and pre-sized to accommodate the relevant anatomical detail of the lower leg and ankle regions of the wearer. One skilled in the art would understand from the description herein that the shape and size of the moldable thermoplastic material 116 may be irregular and/or vary depending on the size, height, and other physiological characteristics of the target wearer. However, the dimensions (as shown in FIG. 4) of the moldable thermoplastic material 116 may be generally derived from benchmarking and anthropometric data, as explained in the example below. The dimensions illustrated in FIG. 4 are not intended is to be limiting, as these values may differ based on one or more physiological characteristics of the wearer (e.g. height, shoe size, weight, etc.). In addition, the shape (as shown in FIG. 4) of the moldable thermoplastic material 116 may be derived from average measurements of the lower leg and malleolus of the wearer, with a height that sits beneath the calf muscle. Based on these data points, the moldable thermoplastic material 116 of brace 100 has an ultimate geometry that allows for ankle stabilization for protection against impact/forces, while minimizing tibia-fibula separation and compressing the target ligaments.

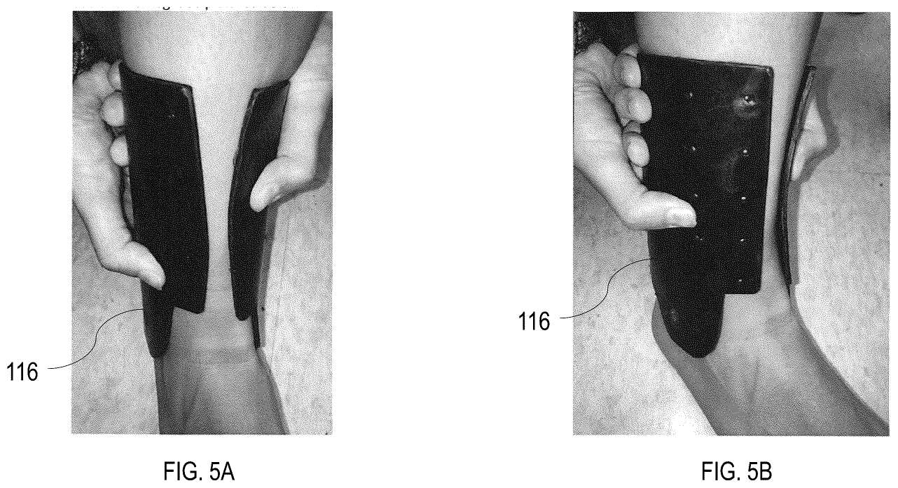

Turning now to FIGS. 5A-5B, assembly of the orthopedic splint 114 includes manipulating a moldable thermoplastic material 116. Generally, the moldable thermoplastic material 116 may be dipped and/or submerged in hot water and may be directly molded in conformity with the wearer's ankle and/or lower leg. Once molded, the orthopedic splint 114 comprising thermoplastic material 116 is inserted into an adjustable pocket 118. As best shown in FIGS. 6A-6B, the adjustable pocket 118 may comprise neoprene material and may be pre-stitched (attached or sewn) to a compression sleeve 120 configured to be wrapped around at least a lower leg of the target wearer. In an exemplary embodiment, the compression sleeve 120 may comprise a breathable polyester compression sleeve. Additionally or optionally, the adjustable pocket 118 includes a hook and loop type mechanism, e.g. a Velcro® strip, disposed toward the wearer's lower leg for minimizing or eliminating any gap or separation between the orthopedic splint 114 and the foot plate 102. Reduction or elimination of this gap allows for a more custom fit and easier integration within the target wearer's shoe. Further, the adjustable pocket 118 comprises a lining having a fabric pattern that is pre-cut and/or pre-sized in accordance with one or more physical or material characteristics (e.g. shape, size, geometry) of a pre-molded thermoplastic material 116 to provide a relatively more snug fit. Achieving this snug fit is generally possible by providing custom fit of as many components of the ankle brace 100, such as the foot plate 102 and the orthopedic splint 114.

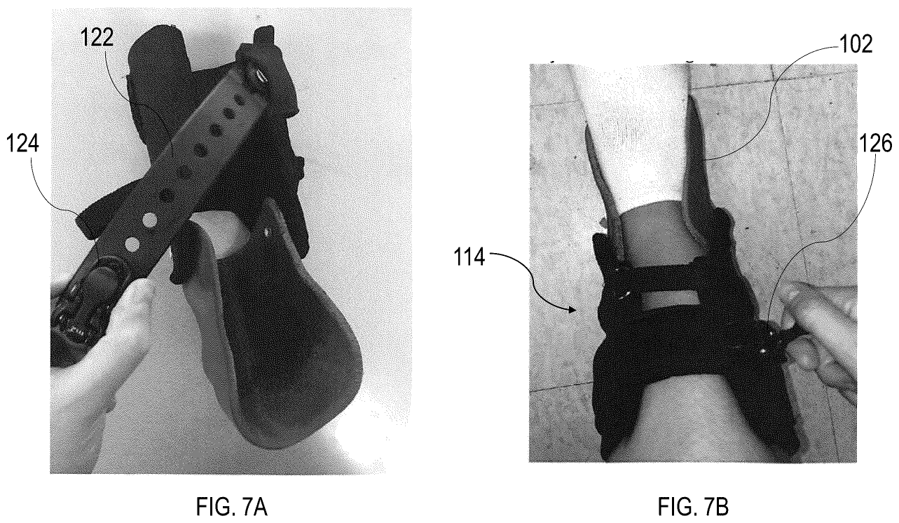

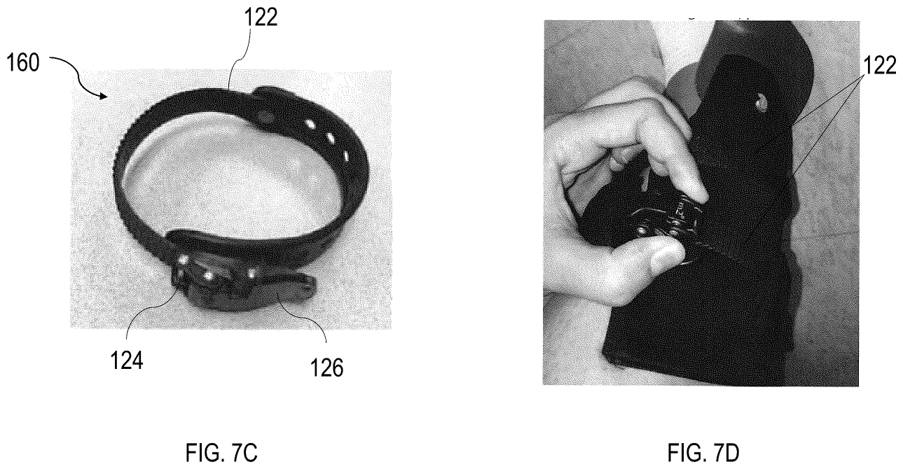

In an exemplary embodiment, the adjustable pocket 118 is configured to adjust compression of the orthopedic splint 114 relative to the lower leg of the target wearer to promote a rehabilitative position of the tibia and the fibula of the target wearer. In one non-limiting example, as illustrated in FIGS. 7A-7D, this adjustment is achieved with the adjustable pocket 118 comprising at least one strap 122, a corresponding buckle 124 for receiving the strap, and a ratchet 126 for adjusting a compression force exerted for securing the orthopedic splint 114 against the lower leg of the target wearer.

The tightening system 160 is formed by the at least one strap 122, the buckle 124, and the ratchet 126 and these components work in concert to secure the ankle brace 100 to the lower leg of the target wearer. The at least one strap 122 may be inserted into a stitched belt loop 132 of an exterior surface of the compression sleeve 120. Further, the buckle 124 may be located on a lateral side of the wearer's leg to avoid discomfort when adjusting the compression force exerted, via tightening the strap 122, for example. This tightening system 160 provides stabilization of the tibia and fibula, thereby providing protection against risk of reinjury. Further, the system allows for easy adjustment, as the user can “crank” the strap 122 to ratchet it to the desired position for comfort and stabilization of the proximal ankle joint. Additionally or optionally, the straps 122 may be secured in place (i.e. locked) into a series of grooves defined by the strap 122 or another component of the brace 110, thereby preventing the strap from inadvertently loosening over time and upon application of impact or force during play. Further, the buckle 124 may be configured to have relatively smaller dimensions (compared to other components of the brace 100, such as the orthopedic splint 114 or the foot plate 102), such that protrusion (extending outwardly from the brace 100) of the buckle 24 is minimized or eliminated.

In another exemplary embodiment, the tightening system 160 may comprise a strap 122, a buckle 124, and a ratchet 126, which are arranged in a configuration mirroring the chinstrap found in helmets (e.g. football helmets). Such systems generally have a low-profile (less bulk and provides greater comfort) and allows for easy and fast adjustment. The buckle 124 in this embodiment is capable of withstanding high impact forces. In yet another exemplary embodiment, the tightening system 160 may additionally comprise padding on the buckle 124 for protection of the buckle 124 from high impact forces. The padding may include one or more layers of elastomeric material configured to provide cushioning from high impact forces encountered during athletic activities, or the like (e.g. exercise). Although the padding may provide extra protection, the padding may undesirably add bulk to the buckle 124, which may increase exposure of operational components of the brace 100 to excessive impact forces. For example, damage to or failure of the buckle 124 may lead to the integrity of the brace 100 being compromised, i.e. inadvertent or unwanted loosening of the brace 100.

The strength and durability (i.e. ability to withstand both long term use and direct impact forces, as well as the repeated range of motion) of this tightening system 160 of the prototype brace 100 was assessed using testing protocols detailed in the example further below. In summary, the assessment involved a weighted drop test to determine whether the ratcheting buckle 124 was able to withstand an impact force, such as the falling force of another player on top of the brace 100. Because at least the is buckle 124 outwardly protrudes to a greater extent than some components of the brace 100, at least the buckle 124 is exposed to the playing environment and thus prone to coming in contact with unwanted falling forces. This exposure required particular consideration because the buckle 124 permits maintaining a rehabilitative position of the tibia and fibula bone, i.e., maintaining the compression of the target ligaments, including the anterior inferior tibiofibular ligament (AITFL) and the posterior inferior tibiofibular ligament (PITFL), which are overlooked in at least existing lateral and medial ankle sprain braces. Accordingly, the buckle 124 of the brace 100 may be configured to withstand an impact force, such that the buckle 124 does not break or otherwise become inoperable upon impact.

Aa load (e.g. 50 lb weight) was dropped on the lateral side of the ankle brace 100, where the buckle 124 may be disposed, for example, and the load was dropped on the buckle 124 from a height of 2 ft and 4 ft. The buckle 124 did not release or loosen when the weight was dropped from 2 ft. The buckle 124 further sustained ratcheting function after the force of the 2 ft drop. Likewise, the buckle 124 also did not release or loosen when the weight was dropped from 4 ft. The buckle 124 also sustained ratcheting function after the force of the 4 ft drop. Accordingly, the buckle was evaluated as effective for both the heights of 2 ft and 4 ft, as there was minimal or no loss in tightening or ratcheting capabilities. Specifically, the buckle 124 was determined to withstand the applied force or load to a factor of safety of 2 (FOS=2). Accordingly, the buckle 124 received a passing score.

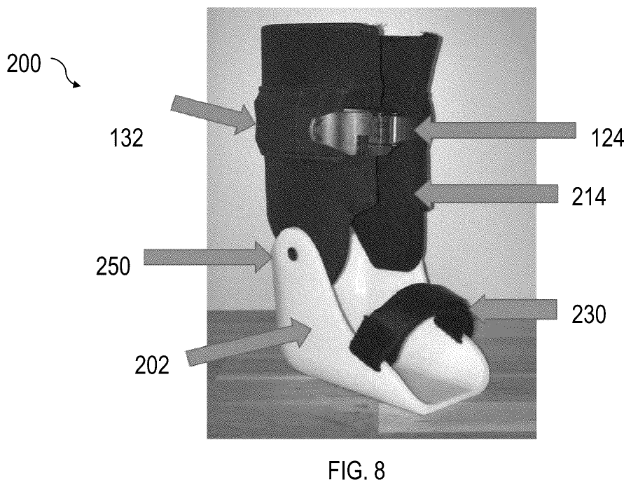

Another embodiment of the brace 100 according to the present invention is illustrated in FIGS. 8-10. The components of this embodiment, such as ankle brace 200, generally correspond to the components of ankle brace 100. However, this embodiment differs from the first embodiment described above in some respects. In one example, ankle brace 200 includes a foot plate 202 pivotably coupled to an orthopedic splint 214 via a hinge mechanism comprising removable heavy duty double capped rivets 250. The foot plate 202 and orthopedic splint 214 are securely coupled to each other, but rivets 250 desirably permits ease of assembly and disassembly of the components of ankle brace 200. On the other hand, the use of rivets 250 on their own to pivotably attach the foot plate 202 to the orthopedic splint 214 may not withstand repeated motion of the foot plate 202 relative to the orthopedic splint 214, especially during rigorous activities, such as sports.

The hinge mechanism of ankle race 200 may further comprise compression fit rivets, such as those utilized in clothing, luggage, and textile applications. These compression rivets comprise two separate pieces: a stem and a cap. They are installed using a rivet press, which applies force or pressure to compress the step and cap together to secure the material(s) placed therebetween. Once the rivet is installed, removing them is not possible with considerable force, thereby desirably increasing the strength and durability of the brace 200. However, this near permanent attachment reduces opportunities for quick disassembly, for cleaning or repair of parts of the brace 100, for example.

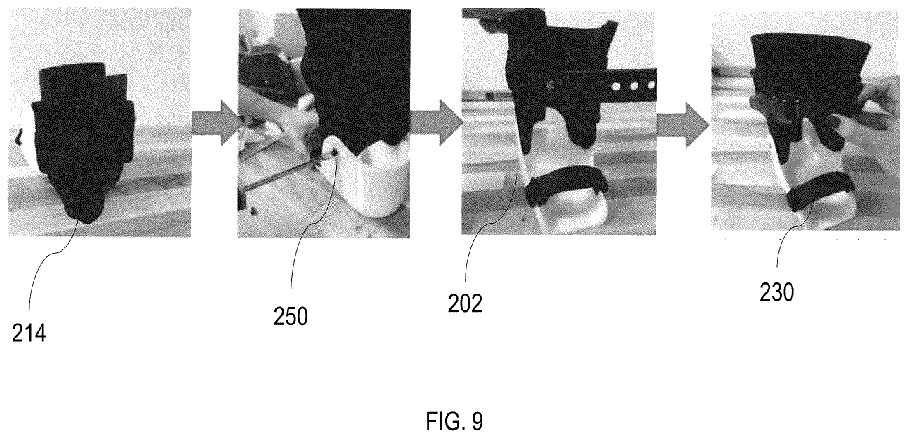

Furthermore, as best seen in FIGS. 9 and 10, the brace 200 includes an adjustable fastening strap 230 connected to the front foot portion of the foot plate 102. The addition of strap 230 creates an opening or space configured to receive the wearer's foot between the fastening strap 230 and the foot plate 202 to compressibly retain the foot of the wearer on the foot plate 202.

### EXAMPLE

The co-inventors assessed the feasibility and functionality of the components of the prototype brace, as well as verified any updates or improvements made. The prototype brace comprised a foot plate and an orthopedic splint pivotably connected to the foot plate. The orthopedic splint is disposed in an adjustable pocket configured to adjust compression of the orthopedic splint relative to the lower leg of the wearer. The prototype was subjected to various mechanical and clinical testing as detailed herein.

For the construction of this prototype, the pre-cut sheet of moldable thermoplastic material of the orthopedic splint was molded to a lower leg and ankle regions of a wearer or subject. After the molding step, the orthopedic splint was inserted into a stitched neoprene pocket lining. The orthopedic splint was hinged to the foot plate portion corresponding to the malleolus of the wearer (when worn) via a screw-in double capped steel rivet and secured using a sealer (Loctite® Threadlocker Red 271™) on the screw. A ratcheting buckle strap was then inserted into the stitched belt loop on the outer lining of the brace, which is used to secure the orthopedic splint around the leg. The fabric pattern for the pocket lining was cut into a shape that directly resembled the pre-molded shape of the orthopedic splint to provide a snug fit. Detailed instructions on how the prototype was assembled can be found below.

### Assembly of Prototype

The following exemplary steps were followed for assembling the prototype.
1. Insert molded orthopedic splint into pocket lining.
2. Drill holes in the footplate. The location of the holes can vary per user. To assist in drilling the holes in the proper location, place the foot plate on the wearer's foot and hold the adjustable pocket with the orthopedic splint therein adjacent the leg. Ask the wearer to perform dorsal and plantar ankle flexion. Find and is mark the pivoting point (see FIG. 3A for example) between the orthopedic splint and the foot plate. Drill a respective hole corresponding to the pivoting point on the lateral and medial wings of the foot plate, each wing covering the lateral and medial malleolus using a 7/32″ drill bit.
3. Insert a binding barrel in the holes on the inside surface of the foot plate.
4. Secure the orthopedic splint to foot plate by placing the binding barrel through the holes in the orthopedic splint.
5. Place a respective washer around the binding barrel on the outside surface of the orthopedic splint.
6. Apply a drop of Loctite® Threadlocker Red 271™ to the binding screw.
7. Screw a respective binding post tightly into the binding barrel.
8. Insert ratcheting buckle strap into belt loop.
9. Adjust the ratcheting buckle and Velcro® strap to desired tightness.
10. To loosen the ratcheting buckle, press on a pair of release tabs on the outside surface of the buckle.

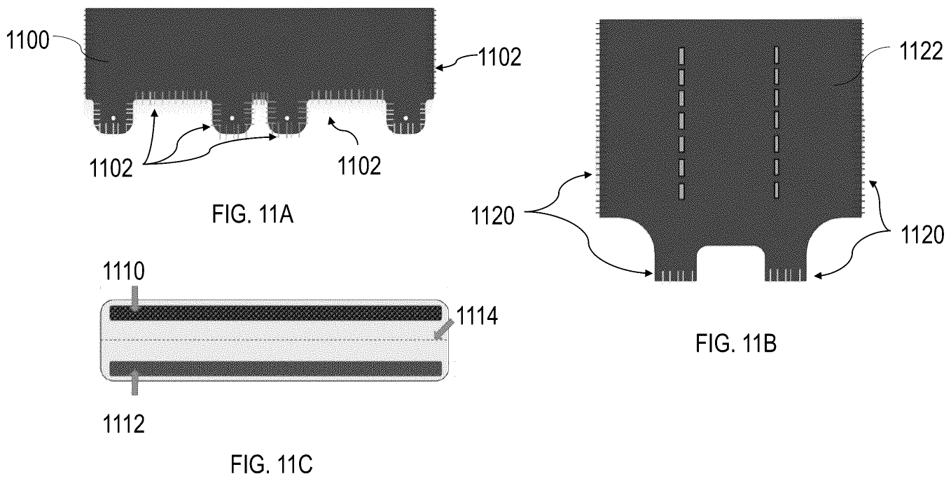

The adjustable pocket lining configured to receive the orthopedic splint of the prototype brace was assembled with reference to FIGS. 11A-11C. FIG. 11A illustrates exemplary stitching 1102 applied to the fabric material 1100 (comprising an elastomer, namely neoprene) used for the adjustable pocket lining. FIG. 11B illustrates exemplary application of the fastening mechanism to retain the orthopedic splint within the pocket lining (e.g. Velcro® strips of microhooks 1110 and microloops 1112 that contact with mating strips inside the pocket when the splint is inserted in the pocket). FIG. 11B also shows central fold line 1114 about which the splint generally bends around the wearer's lower leg, although it should be understood that the bend is generally gradual with a gradient of bending from the centerline to each fastener strip. FIG. 11C illustrated exemplary stitching patterns 1120 for connecting the neoprene pocket to compression sleeve 1122 for creating the pocket lining for the adjustable compression sleeve.

Size Chart for Ankle Brace

| | | Ankle | Foot | Foot | Foot | | |
|---|---|---|---|---|---|---|---|
| | Ankle | Circum- | plate | plate | plate | Splint** | Splint** |
| Shoe | Brace | ference | Width | Height | Length | height | length |
| Size* | Size | (in.) | (in.) | (in.) | (in.) | (in.) | (in.) |
| 5-7 | XS | 11 | 3.50 | 4.5 | 5.5 | 5 | 10 |
| 8-10 | S | 12 | 3.82 | 4.5 | 6.15 | 5 | 11 |
| 11-13 | M | 13 | 4.14 | 4.5 | 6.75 | 5.5 | 12 |
| 14-16 | L | 14 | 4.46 | 4.5 | 7.31 | 5.5 | 13 |
| 17-19 | XL | 15 | 4.77 | 4.5 | 7.69 | 6 | 14 |

*male shoe sizes
**moldable thermoplastic material, e.g. ORTHOPLAST ®

The prototype ankle brace conformed to the dimensions of a young woman or male shoe size 6, which corresponds to ankle brace size XS per the chart above. In general, dimensions of the brace may be customized for the wearer by reference to anthropometric data charts documenting body segment length as a function of total height, which are known in the art, or by reference to actual dimensions of the wearer as measured. The dimensions were extrapolated based on the initial measurements of the dimension of the size XS prototype ankle brace. The ankle brace standard sizes is (XS, S, M, L, XL) were chosen to correspond to shoe size for ease of size determination for male athletes. The male shoe size range was broken into 5 groups and correlated to brace size from XS to XL. Brace size was related to ankle circumference through the standard benchmarking of sizes already on the market, such as those found in U.S. Pat. No. 6,024,712. The footplate width was calculated by finding the diameter of the ankle joint when the ankle circumference was known. The circumference was divided by π to calculate the diameter used as the standard for footplate width. The height of the back of the foot plate (i.e. heel cup) and height of malleolus hinge connectors (e.g. rivets) was held constant. For example, the average ankle malleolus height is between 3.5-4.5 inches. The middle shoe size length for each brace size was used to determine the foot plate length. After the prototype was determined to be an XS based off a shoe size of 6, the foot plate length was measured to be 60% of total size shoe length. The rest of the footplate lengths were calculated by taking 60% of the foot bed length of the middle shoe of the range for the size. The height of the back part of the splint, which covers the proximal Achilles tendon, was determined, in part, through anatomical muscle marks. The initial height of 5 inches on the prototype was determined so it covered a majority of distal shaft, but not high enough to come in contact with the bulk of the calf muscle. This height was confirmed for smaller two sizes and the height increases by a half an inch higher for M and L sizes and a full inch higher for XL size. Finally, the length of the splint is similar to ankle circumference as once the moldable thermoplastic material is molded, the length wraps around the ankle in conformity with the ankle circumference. A 1-inch gap between the two ends of the thermoplastic material was determined to be standard across all braces, to allow for adjustment (e.g. tightening or adjustment of compression force exerted in securing the split against the lower leg of the wearer). Thus, to determine splint length, 1 inch was subtracted from the ankle circumference for each size group.

Engineering assessments and analyses performed or suggested to be is performed using the prototype brace will now be discussed below. These assessments were informed by target metrics previously established by the co-inventors, in an effort to address the limitations of the existing ankle braces for care and management of ankle injuries (e.g. ankle sprains). Such efforts included an assessment of the limitations of prior art designs and of feedback from target groups or customers (e.g. wearers of the ankle brace device or system), as detailed in Tables 1 and 2 below.

TABLE 1
Exemplary Embodiment Design Constraints

| Constraint | Description | Target Value or Metric |
|---|---|---|
| Durability | Able to withstand long term use, wear | Withstand 300 pounds over |
| | and tear, and maintain functionality | mechanical loading cycles |
| | over time | that are experienced over a |
| | | sports season |
| | | (approximately 5 months) |
| Range of | Minimize internal/external rotation of | <15° of rotation |
| Motion | ankle joint | |
| Stability | Able to stabilize and reduce separation | >3 on Questions |
| | of the tibia and fibula | 5 and 6 of survey |
| Adjustability | Able to be adjusted for a (custom) fit | Sizes (XS-XL) |
| | to the individual | Pass/Fail |
| Size | Able to be worn within a shoe | Pass/Fail |
| Cost-Effective | Able to be produced at a reasonable | <$400 |
| | cost to accommodate customers, such | |
| | as athletes or universities | |
| Compliance | Follows uniform regulations regarding | |
| | protective equipment, such as those | |
| | provided by the NCAA, | |

The following justifications detail how the target values for each design constraint of the exemplary embodiment were established. For example, metrics and justifications were generated using user surveys, engineering standards, patents, benchmarking, and the requests of the sponsors.

An exemplary metric for durability may include the ability to withstand the wear and tear of athletic competition, including (ideally) lasting at least an entire sports season. The recovery timeline for a high ankle sprain can be anywhere from six weeks to six months long, and so it is important to properly manage the care of the injury throughout this recovery time. Therefore, maintaining the integrity and full functionality of the inventive high ankle brace is a target in protecting and stabilizing the proximal ankle joint after repeated use by the wearer. In this regard, an adapted testing protocol to measure mechanical durability and for the conduction of fatigue testing was established using the Standard American Society for Testing and Materials is (ASTM) F1900, as described in ASTM F1900-98(2017) Standard Test Method for Water Resistance of Footwear Using a Walking Step Simulator, ASTM International, West Conshohocken, PA, 2017, incorporated herein by reference, and Standard ASTM F1976, as described in ASTM F1976-13 Standard Test Method for Impact, incorporated herein by reference, and Attenuation of Athletic Shoe Cushioning Systems and Materials, ASTM International, West Conshohocken, PA, 2013, incorporated herein by reference.

Adaptations of these standard testing procedures included increasing the load to 300 pounds (lbs.) because of the intended or target wearer of the ankle brace, such as a football player. As an example, it is assumed that football players take a maximum of 60-70 repetitions per game, which was used to calculate mechanical loading cycles corresponding to a 5-month sport season. On an average play, a lineman typically takes 5-6 steps engaged with another player. Further, due to the higher levels of intensity against opponents and increased risk of impact to the injured ankle or to the player generally, the primary focus was assessment of the functionality during play or participation in athletic activities (e.g. games or sports). However, one skilled in the art would understand from the description herein that use of the ankle brace is not limited during play, but instead may be used additionally or optionally during practice games or training sessions. Ultimately, with a typical college football season being 12 games long, for example, the ankle brace must be sufficiently durable to withstand at least 4,320-5,040 cycles of use or impact.

To assess range of motion, minimizing the inversion/eversion of the ankle joint while still allowing plantar/dorsiflexion motion is optimal. The degrees for ankle range of motion were determined based on a physical therapy study, which established ideal degrees of motion for healing a high ankle sprain. Two prior art treatment methods, tape and lace up ankle braces, provide a range of motion between 26-33 degrees. Therefore, an exemplary target value of less than 15 degrees of internal and external rotation was established to provide a desired degree of stability in the exemplary embodiment.

In an exemplary metric for stability, the tibia and fibula bones ideally do not separate upon performance of weight bearing activities and competitive play. A is prototype device used by the wearer was evaluated using a survey question and response. In satisfaction of this metric, the score assigned in Questions 5 and 6 should be higher than a 3 out of 5, indicating that the user agrees or strongly agrees that the prototype device provides adequate ankle stability and that they feel confident while wearing it during play or athletic activity. One skilled in the art would understand from the description herein that a quantitative measure of the separation or gap at the distal tibiofibular syndesmosis can be evaluated by other testing protocols, such as X-ray and CT scans.

In an exemplary metric for adjustability, a relatively more personalized fit for the wearer may be achieved via one or more components that allow for tightening and adjustments for comfort. Additionally or optionally, the ankle brace may be provided in standard incremental sizes (XS, S, M, L, XL, 2XL) which correspond to US shoe sizes, to obtain a custom fit. In this way, a score of “pass” was awarded if the ankle brace has at least one adjustable component and can be produced in various sizes.

In an exemplary metric for size, the high ankle brace includes a low profile so as to not hinder the performance of the athlete. For example, the brace is easily integrated and can fit comfortably in a wearer's normal footwear, e.g. shoe or cleat. This integration is evaluated according to a standard of not having more than a 1 size increase in accordance with ASTM Standard F539-01(2017), as described in ASTM F539-01(2017) Standard Practice for Fitting Athletic Footwear, ASTM International, West Conshohocken, PA, 2017, incorporated herein by reference. Additionally or optionally, the size of the brace may be consistent with the size chart of ankle circumference described in U.S. Pat. No. 6,024,712. Thus, a “pass” score was awarded if the brace fit correctly into a shoe and was of appropriate size for the wearer.

An exemplary metric for cost-effectiveness may include adopting a target cost threshold that allows athletes at different levels of competition and resources to utilize embodiments of the invention. While a target of <$400 was selected for a University athlete, it should be understood that lower thresholds may be established for athletes with less resources, and higher thresholds for professional athletes with greater resources. One exemplary prototype had a total materials cost of <$70 (meaning that a commercial price could be set at 2× or 3× and still be considered cost effective).

In an exemplary metric for compliance, embodiments of the ankle brace may optimally comply with the relevant regulations of the subject athletic activity or sport, such as the NCAA 2016 AND 2017 RULES AND INTERPRETATIONS, and is subsequent versions thereof, incorporated herein by reference. For example, the brace ideally does not endanger other players, or project hard and abrasive materials during use, and is eligible for approval by an official or sports professional before use in play or competition.

TABLE 2
Wearer desirables

| | | Exemplary Target |
|---|---|---|
| Desirable | Description | Value or Metric |
| Comfort | Does it create additional discomfort | >3 on Questions 1 and 2 of |
| | to the wearer? | survey |
| Pain Reduction | Does it lessen the pain of the injury | <4 on Question 8 of survey |
| | | and a decrease of at least 2 |
| | | from Question 7 to Question 8 |
| Ease of Use | Is it easy to put on and take off by | 5 minutes on Question 3 of |
| | the wearer | survey |
| Lightweight | Is it constructed from a light, low | <12 ounces |
| | profile material so as to not obstruct | |
| | player performance | |
| Cleanable | Is it machine washable or easily | Pass/Fail |
| | cleaned after use | |
| Aesthetics | Is it visually appealing | >3 on Question 4 of survey |

A survey may be conducted with questions (discussed in more detail below) directed to wearers regarding each of the Desirables/Wants shown in Table 2. Survey scores indicating that the users agree or strongly agree that the brace allows them to compete with functional pain, and perform at a high level without experiencing excessive blistering/additional discomfort with repeated use are ideal.

Pain reduction facilitated by the ankle brace may be evaluated using a visual analog pain scale. Scores demonstrating moderate to no pain with the use of the brace, and that the athlete's performance was not significantly hindered by pain caused by the high ankle injury are ideal. One of ordinary skill in the art would understand from the description herein that effectiveness of the ankle brace in pain reduction does not require eliminating pain altogether, and varying degrees of pain tolerance by the target wearer can be achieved.

Regarding ease of use, scores indicating that the user agreed or strongly agreed that the device was easy to use are desired.

A brace that is lightweight is desired for maintaining athletic performance because a relatively heavier brace may hinder mobility. For example, one target for an exemplary prototype device may be a weight of less than 12 ounces. Heavier or lighter thresholds may alternatively adopted. For example, one exemplary prototype had a weight of between 16-17 ounces.

A brace that is cleanable includes materials that are machine washable or cleanable with household products. A cleaning validation approach for medical devices, as described in ASTM F3127-16 Standard Guide for Validating Cleaning Processes Used During the Manufacture of Medical Devices, ASTM International, West Conshohocken, PA, 2016, incorporated herein by reference, was adapted to ensure that exemplary embodiments of the brace were able to be cleaned with suitable chemicals that are not overly harsh/abrasive on the wearer's skin or the brace itself.

As for aesthetics, scores indicated that the wearer agrees or strongly agrees that the brace is aesthetically pleasing.

### Exemplary FEA Analysis of Foot Plate

The co-inventors conducted a Finite Element Analysis (FEA), as part of the SOLIDWORKS® Simulation structural analysis tools, of the prototype brace design to assess the behavior of components or assemblies of the brace when a load is applied. The applied load may comprise pressure, force, temperature, gravity, centrifugal loads. In particular, in the FEA, the applied loads simulated the internal and external forces that the brace experiences during mobility, such as when worn by a wearer, and in impact situations that increase risk of high ankle sprains, including but not limited to participation in contact sports or other athletic activities. An example calculation of the internal and external forces may include internal and external forces experienced at the ankle region when an athlete or player falls directly on to the knee region of another athlete or player.

Relatively higher stresses and potential for points of failure of the prototype brace design may be located at the malleolus of the foot plate, where the brace is hinged. Two different foot plate designs were assessed via FEA of the hinge joint between the respective foot plate designs and orthopedic splints to determine the configuration with the more desirable mechanical properties. Specifically, the loads applied in the FEA simulated the forceful external rotation of the foot during impact (i.e. during participation in athletic activities or contact sports), which may increase the risk of high ankle sprains or injuries.

Prototype design (a) features a relatively low-cut heel plate, and prototype design (b) includes medium heel plate, i.e. enclosed heel plate that extends just past the calcaneus (heel bone).

The results of the FEA suggests that the enclosed calcaneal heel plate (b) more effectively distributes the simulated loads, as it exhibited comparatively lower von Mises stress values, i.e. heel plate (b) demonstrated a lower maximal stress value of 5.7 E8 N/m\hat{ }2 as compared to the maximal stress value of the low-cut or open heel plate (a), which was 6.869 E8 N/m\hat{ }2. Thus, the co-inventors determined that a brace having an enclosed calcaneal heel plate (b) provided more desirable stabilization of the ankle joint, while simultaneously reducing risk of failure.

Exemplary Testing Protocol for Fatigue Testing Pivotable Connection (e.g. Hinge Joint)

The protocol assessed whether the hinge joint design between the foot plate and the orthopedic splint of the prototype brace can withstand repeated rotation that is associated with movement (e.g. running) during participation in an athletic activity for a duration of time, e.g., for a full football season. This testing protocol was adapted from a snowboard binding test that uses a linear actuator. A passing score for this durability metric showed the pivotable connection (e.g., a hinge) does not break or become inoperable after repeated articulation (e.g., forward and backward movement) of the foot plate relative to the orthopedic splint.

Procedure: The prototype brace may be tested in a linear actuator machine, or manually (as in this case). The following exemplary steps were followed for assessing durability of the prototype hinge joint.

1. Insert brace into athletic shoe and tighten to prosthetic cylinder.

2. Connect brace-cylinder complex to rod on end of linear actuator.

3. Fix shoe to ground so that it will not move when force from linear actuator is applied.

4. Connect linear actuator to power source and power on.

5. Set linear actuator to move 6.5 inches and back to start, such that the hinge joint will flex from a plantar flexed position of 11 degrees and move to a dorsiflexed position of 7 degrees, which is representative of the running motion of a braced ankle joint. See Cordova, M. L., Ingersoll, C. D., & LeBlanc, M. J. (2000). Influence of Ankle Support on Joint Range of Motion Before and After Exercise: A Meta-Analysis. Journal of Orthopaedic and Sports Physical Therapy.

6. Begin with brace-cylinder complex in a 101 degree angle with the ground, such that the hinge represents plantar flexion.

7. Run linear actuator for 4,320-5,040 repetitions to simulate the rotations on the hinge for a 5-month sport season. The number of repetitions, was calculated based off the average number of steps a lineman takes per rep (5-6), the average number of is reps per game (60-70) and a season comprising 12 games.

8. Stop the machine if the hinge breaks off, unscrews, falters, or becomes inoperable in any way.

9. After all repetitions are complete, power off the linear actuator and remove the brace from the set up. Evaluate the hinge joint for any deformations. The brace will receive a passing score if no deformations or breakages are seen.

### Exemplary Testing Protocol for Buckle Impact Test (Drop Test)

The protocol assessed whether the buckle receiving a corresponding strap can withstand an impact simulating a human (e.g. athlete, or someone else other than the wearer of the porotype brace) falling upon the prototype brace. A passable buckle or mechanism for adjusting the compression force exerted for securing the orthopedic splint against the lower leg does not break, loosen, detach, or becomes inoperable after application of the impact or force.

Procedure: The prototype brace was tested with a weighted sandbag for simulating an impact, force, or shock resulting from a free fall, e.g. contact with another athlete during play. The following exemplary steps were followed for assessing durability of the prototype buckle.

1. Tighten buckle around prosthetic cylinder.

2. Position buckle complex on ground and secure with stakes if needed to prevent unnecessary motion.

3. Obtain 50 lb plate weight. Double check weight on scale. A 50 lb plate weight was chosen for ease of obtainment as a 300 lb weight would not be practical to hold. The 50 lb weight was used to figure out at what drop height the impact force would be equivalent to 1334 N (300 lbs). The plate was wrapped in cloth fabric to create a very thin shock absorber to the force.

4. Hold bag 2 ft above the ground directly over the buckle. The calculations below determined the height from which the 50 lb weight plate needed to be dropped from in order to simulate a 300 lb impact force on the buckle.

5. Drop weighted bag onto buckle and then remove.

6. Tighten the strap around test cylinder and evaluate effectiveness.

7. If effective, repeat the trial at a height of 4 ft to evaluate for a factor of safety of 2.

8. If effective again, repeat the trial at a height of 6 ft to evaluate for a factor of safety of 3.

### Prophetic Exemplary Testing Protocol for Fatigue Testing on Walk-Step Simulator

The following protocol may be used for assessing the durability of the entire is ankle brace by simulating the running steps during a full season of a sport or athletic activity. This test was adapted using the ASTM F1900, as described in ASTM F1900-98 (2017) Standard Test Method for Water Resistance of Footwear Using a Walking Step Simulator, ASTM International, West Conshohocken, PA, 2017, incorporated herein by reference. An exemplary brace is considered to receive a passing score, if it does not break, falter, or become inoperable for all of the repetitions.

Procedure: Attach prototype brace to a prosthetic limb and run through a Walk-Step simulator that is common in athletic shoe testing. Set load of machine to 300 lbs, equivalent to the weight of a football lineman. Set speed of machine to 150 steps/min, equivalent to a fast run. Run machine for 28-34 min, the equivalent of approximately 4,320-5,040 repetitions to simulate a 5-month football season. The number of cycles, 4,320-5,040, was calculated based off the average number of steps a lineman takes per rep (5-6), the average number of reps per game (60-70) and a season comprising 12 games. After all repetitions, remove brace from machine and check for breakage. Brace may be given a passing score, for example, if after evaluation the brace has no structural faults or broken components.

### Prophetic Exemplary Testing Protocol for Impact Testing on Brace at Maximum Load

The protocol is intended to assess whether the prototype brace can withstand the force of an instantaneous impact during play or athletic activity, e.g., a tackle from another player or athlete. This protocol is based on a universal compression test to determine impact strength. An exemplary passing score of the prototype brace may show, for example, that the brace withstand the loads applied, with a factor of safety of 3.

Procedure: The prototype brace can be set up to run in a universal compression testing machine, as described and designed by Instron of Norwood, Massachusetts.

1. Insert a prosthetic cylinder into the ankle brace and fully tighten around leg.

2. Shift the position of the ratcheting buckle slightly anterior such that the upper head of the Instron compressor does not come in contact with it when placed horizontal into the machine.

3. Clamp leg-brace complex to an Instron compressive such that the medial side of the orthopedic splint is lying on the lower compressive head of the Instron compressor. The brace is placed lying down to simulate the greatest sideways impact the brace could experience from a force or impact during play, e.g., a tackle. If the brace was placed at an angle, some of the force would be distributed vertically, thus decreasing the horizontal vector. The test assumes that if the brace can withstand the greatest horizontal impact, then a force or impact, e.g., a tackle, made from a position higher than the location of the ankle brace could also been withstood.

4. Ensure the upper head of the Instron compressor will come in contact with the brace at the lateral side of the orthopedic splint and not distally at the hinge or foot plate. The compression head should be placed on the orthopedic splint and not at the hinge or footplate because a sideways tackle would not be able to be distal enough in order to come in contact with the footplate that is hidden within the shoe. It is more logical to place the compression head higher up the brace to simulate a more realistic sport scenario or athletic activity

5. Load the Instron machine for a single impact cycle (loading and unloading) of 900 lb (4000 N). This ensures a factor of safety of 3 for the load of a 300 lb force falling on the brace during play or athletic activity.

6. Continue running the test until the load reaches 4000 N or until failure.

7. Export test data to generate a force vs. displacement curve.

8. Remove brace from Instron machine and evaluate for deformations.

9. The brace may be given a passing score if, for example, the brace does not deform, crack, soften, or otherwise become inoperable after the load has been completed.

### Exemplary Metric Survey

Numerous survey questions were developed to evaluate the success of the final product with respect to the constraints/desirables outlined in Tables 1 and 2. This survey was designed to be answered by the wearer.

Target wearers were asked to rank level of agreement with statements on a scale of 1—5 as indicated below, with 5—Strongly agree, 4—Agree, 3—Neither agree nor disagree, 2—Disagree, and 1—Strongly Disagree. Exemplary questions included:

1. This product allows me to compete with functional pain, and does not hinder my performance when tightened fully and worn during activity.

2. This product does not cause excessive blistering of the skin or other additional pain/discomfort, not associated with the high ankle sprain, after repeated use.

3. This product was easy to put on and tighten correctly in less than 5 minutes and did not require assistance from an athletic trainer.

4. This product is aesthetically pleasing and I would be satisfied wearing it.

5. During activity, this product improved the stability of my ankle joint as compared to not wearing the brace.

6. I feel more confident during competitive play and feel that my injured ankle will hold up with the aid of this brace.

For the following questions, users are asked to rank the severity of their pain on a 10-point scale known in the art, with 10=unable to move, and 0=no pain.

7. Without the brace, I experience this amount of pain due to my high ankle sprain when playing sport.

8. When wearing that brace, I experience this amount of pain due to my high ankle sprain while playing sport.

It should be understood that all of the testing protocols as described above are merely exemplary, and that suitable embodiments for some purposes may not meet all constraints or desirables or may have varying degrees of performance relative to the foregoing factors.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. An ankle brace configured to be worn by a target wearer, the target wearer having a foot, an ankle, a lower leg, and a tibia and a fibula disposed within the lower leg, the ankle brace comprising:
a foot plate having a foot bed contoured to underlie the foot of the target wearer, the foot bed having a heel cup extending generally upward from the foot bed and along a posterior aspect of the ankle of the target wearer;
an orthopedic splint pivotably coupled to the foot plate and configured to at least partially wrap around the lower leg of the target wearer, the orthopedic splint disposed in an adjustable pocket, the adjustable pocket configured to adjust compression of the orthopedic splint relative to the lower leg of the wearer to promote a rehabilitative position of the tibia and the fibula of the target wearer.

2. The ankle brace of claim 1, wherein the heel cup comprises a first set of material properties and a front foot portion of the foot bed comprises a second set of material properties that is different than the first set of material properties.

3. The ankle brace of claim 2, wherein the heel cup has relatively greater flexibility than the front foot portion of the foot bed.

4. The ankle brace of claim 1, wherein the foot plate comprises a medial wing extending generally upward from the foot bed and along a medial aspect of the ankle of the target wearer, the foot plate also including a lateral wing extending generally upward from the foot bed and along a lateral aspect of the ankle of the target wearer.

5. The ankle brace of claim 4, wherein the medial wing extends over the medial malleolus of the ankle of the target wearer and the lateral wing extends over the lateral malleolus of the ankle of the target wearer.

6. The ankle brace of claim 4, wherein the medial wing and the lateral wing are each extending along an interior surface of the orthopedic splint.

7. The ankle brace of claim 1, wherein a front foot portion of the foot bed defines a front edge that does not extend beyond metatarsal bone heads of the target wearer.

8. The ankle brace of claim 7, wherein the front edge is disposed within an area defined between the metatarsal bone heads of the target wearer and tarsometatarsal joints of the target wearer.

9. The ankle brace of claim 4, wherein the lateral wing, the medial wing, the heel cup, and the foot bed collectively form an open section between a front ankle portion and a front foot portion for facilitating mobility of the target wearer.

10. The ankle brace of claim 9, wherein the lateral wing, the medial wing, the heel cup, and the foot bed collectively form an open section between a front ankle portion and a front foot portion for facilitating an articulation of the foot plate relative to the orthopedic splint.

11. The ankle brace of claim 9, wherein the front ankle portion and the front foot portion are positionable to avoid contact with the brace.

12. The ankle brace of claim 1, wherein the orthopedic splint comprises a moldable thermoplastic material.

13. The ankle brace of claim 1, where in the heel cup extends above and encloses a calcaneus of the target wearer.

14. The ankle brace of claim 1, wherein the adjustable pocket comprises at least one strap, a corresponding buckle receiving the strap, and a ratchet for adjusting a compression force exerted for securing the orthopedic splint against the lower leg of the target wearer.

15. The ankle brace of claim 14, wherein the buckle is positionable on a lateral side of the leg of the target wearer.

16. The ankle brace of claim 14, wherein the strap may be releasably attached to a plurality of grooves defined by the strap.

17. The ankle brace of claim 1, further comprising an adjustable fastening strap connected to the front foot portion and the ankle brace is configured to receive the foot of the target wearer between the fastening strap and the foot plate to compressibly retain the foot of the target wearer on the foot plate.

18. The ankle brace of claim 1, wherein at least the foot plate is sized to be inserted in a shoe of the target wearer.

19. A process for making the ankle brace of claim 1, the process comprising the steps of:
accepting a plurality of customization inputs, the input comprising a plurality of parameters corresponding to an insole of the shoe of the target wearer;
constructing a digital model for characterizing the foot plate;
transmitting the digital model to a computer aided manufacturing process for automatically fabricating the ankle brace.

20. The process of claim 19, wherein constructing the digital model comprises capturing an image of the foot or the ankle of the target wearer.

21. The process of claim 20, wherein constructing the digital model comprises sculpting the foot plate around the captured image of the foot or ankle of the target wearer, including defining a front edge of a foot portion of the foot bed that does not extend beyond metatarsal bone heads of the target wearer, and defining at least a portion of a periphery of the heel cup using a curve that extends around the ankle proximal to metatarsal bone heads, but not beyond a tarsometatarsal joint, around a medial malleolus of the ankle and a lateral malleolus of the ankle of the target wearer, such that at least a calcaneus of the target wearer is enclosed.

22. The process of claim 21, wherein portions of the curve around the medial malleolus of the ankle of the target wearer and lateral malleolus of the ankle of the target wearer are generally symmetrical.

23. The ankle brace of claim 1, wherein the foot plate is customized to the target wearer based upon a digital model populated with a plurality of parameters corresponding to customization inputs defined by an insole of the shoe of the target wearer.

24. The ankle brace of claim 23, wherein at least some of the plurality of parameters in the digital model correspond to customization inputs derived from a captured image of the foot or ankle of the target wearer.

25. The ankle brace of claim 24, wherein at least a portion of a periphery of the heel cup is defined by a curve sculpted relative to a foot and ankle geometry of the target wearer as derived from the capture image, the curve extending around the ankle, proximal to metatarsal bone heads, but not beyond a tarsometatarsal joint, and around a medial malleolus of the ankle of the target wearer and a lateral malleolus of the ankle of the target wearer, such that at least a calcaneus of the target wearer is enclosed.

26. The ankle brace of claim 25, wherein portions of the curve around the medial malleolus of the ankle of the target wearer and lateral malleolus of the ankle of the target wearer are generally symmetrical.

27. An ankle brace comprising a product of the process according to claim 19.
